(19) United States
(12) Patent Application Publication
(10) Pub. No.: US 2022/0313910 A1
(43) Pub. Date: Oct. 6, 2022

(54) AUTOMATED SYSTEM FOR CONTROLLING BLOOD SUGAR LEVELS

(71) Applicant: Commissariat à I'Énergie Atomique et aux Énergies Alternatives, Paris (FR)

(72) Inventors: Eléonore Maeva Doron, La Tronche (FR); Gaelle Ardito, Grenoble (FR); Emma Villeneuve, Grenoble (FR)

(73) Assignee: Commissariat à I'Énergie Atomique et aux Énergies Alternatives, Paris (FR)

(21) Appl. No.: 17/629,335

(22) PCT Filed: Jul. 17, 2020
(86) PCT No.: PCT/EP2020/070238

§ 371 Date: Jan. 21, 2022

(30) Foreign Application Priority Data
Jul. 25, 2019 (FR) 1908457

(51) Int. Cl. A61M 5/172; G16H 20/17

(52) U.S. Cl. CPC A61M 5/1723; G16H 20/17; A61M 2230/201; A61M 2205/50

(57) ABSTRACT

A blood glucose regulation system including a processing and control unit configured to implement an automated blood glucose regulation method, wherein the regulation method takes into account at least one hyperparameter having a default value, the value of said at least one hyperparameter being adjustable on the fly by the processing and control unit by means of an adjustment function, and wherein the processing and control circuit is configured to, after a regulation period: estimate the performance of the regulation method by means of at least one performance indicator; and adjust on the fly the value of said at least one hyperparameter according to said at least one performance indicator.

The present patent application claims the priority benefit of French patent application FR19/08457, which is herein incorporated by reference.

### TECHNICAL BACKGROUND

The present description relates to the field of automated blood glucose regulation systems, also called artificial pancreases.

### PRIOR ART

An artificial pancreas is a system enabling to automatically regulate the insulin inputs of a diabetic subject or patient based on their glycemia (or blood glucose) history, on their meal history, on their insulin injection history.

Examples of regulation systems of this type are particularly described in international patent applications No WO2018/055283 (DD16959/B15018), No WO2018/055284 (DD17175/B15267), and No WO2019/016452 (DD17609/B15860), and in French patent applications No 18/52354 of Mar. 20, 2018 (DD18479/B16770), No 18/56016 of Jun. 29, 2018 (DD18587/B16893), No 18/00492 of May 22, 2018 (DD18480/B16894), No 18/00493 of May 22, 2018 (DD18588/B16895), and No 18/73812 of Dec. 21, 2018 (DD18986/B17521), previously filed by the applicant.

It would be desirable to at least partly improve certain aspects of known artificial pancreases.

### SUMMARY

Thus, an embodiment provides a blood glucose regulation system comprising a processing and control unit configured to implement an automated blood glucose regulation method,

wherein the regulation method takes into account at least one hyperparameter having a default value, the value of said at least one hyperparameter being adjustable on the fly by the processing and control unit by means of an adjustment function,
and wherein the processing and control circuit is configured to, after a regulation period:
estimate the performance of the regulation method by means of a performance indicator; and
adjust on the fly the value of said at least one hyperparameter according to the value of said at least one performance indicator.

According to an embodiment of the present invention, the system further comprises:

a blood glucose sensor; and

an insulin injection device,

and the blood glucose regulation method implemented by the processing and control unit comprises the control of the insulin injection device by taking into account measurements provided by the blood glucose sensor.

According to an embodiment of the present invention, the performance indicator is an indicator from the group comprising:
a percentage of time past in hypoglycemia or a number of passages in hypoglycemia during the regulation period;
a percentage of time past in hyperglycemia or a number of passages in hyperglycemia during the regulation period;
a percentage of time past in normoglycemia during the regulation period;
a quantity representative of the variability of the blood glucose during the regulation period; and
a number of glucose administrations recommended to the user during the regulation period.

According to an embodiment of the present invention, the value of said at least one hyperparameter is kept constant by the processing and control unit during the regulation period.

According to an embodiment of the present invention, said at least one hyperparameter is used a plurality of times by the processing and control unit during the regulation period.

According to an embodiment of the present invention, said at least one hyperparameter is used at least twenty times by the processing and control unit during the regulation period.

According to an embodiment of the present invention, said at least one hyperparameter is a hyperparameter from the group comprising:
a blood glucose threshold below which the user is considered as being hypoglycemic by the processing and control unit;
a coefficient used by the processing and control unit to determine the volume of insulin doses to be injected to the user;
a tolerated threshold of error between a blood glucose prediction made by the processing and control unit by means of a mathematical model, and the real blood glucose measured by the sensor;
a period of inhibition between two successive blood glucose administration recommendations made to the user by the processing and control unit; and
a value of increase of a blood glucose target, applied by the processing and control unit before a physical activity declared by the user.

According to an embodiment of the present invention, said at least one hyperparameter is the coefficient used by the processing and control unit to determine the volume of insulin doses to be injected to the user, and said at least one performance indicator is the percentage of time past in hyperglycemia or the number of passages in hyperglycemia during the regulation period,

and, at the step of adjustment on the fly of the value of said at least one hyperparameter according to said at least one performance indicator, the processing and control unit increases the value of the coefficient if the value of the indicator is greater than a threshold.

### BRIEF DESCRIPTION OF THE DRAWINGS

The foregoing features and advantages, as well as others, will be described in detail in the following description of specific embodiments given by way of illustration and not limitation with reference to the accompanying drawings, in which:

FIG. 1 schematically shows in the form of blocks an example of an automated system for regulating a patient's blood glucose according to an embodiment; and

FIG. 2 illustrates an example of an automated method for adjusting a blood glucose regulation method capable of being implemented by the system of FIG. 1.

### DESCRIPTION OF THE EMBODIMENTS

Like features have been designated by like references in the various figures. In particular, the structural and/or functional features that are common among the various embodiments may have the same references and may dispose identical structural, dimensional and material properties.

For the sake of clarity, only the steps and elements that are useful for an understanding of the embodiments described herein have been illustrated and described in detail. In particular, the blood glucose measurement devices and the insulin injection devices of the described regulation systems have not been detailed, the described embodiments being compatible with all or most known blood glucose measurement and insulin injection devices. Further, the hardware implementation of the processing and control unit of the described regulation systems has not been detailed, the forming of such a processing and control unit being within the abilities of those skilled in the art based on the functional indications of the present disclosure.

Unless specified otherwise, the expressions “around”, “approximately”, “substantially” and “in the order of” signify within 10%, and preferably within 5%.

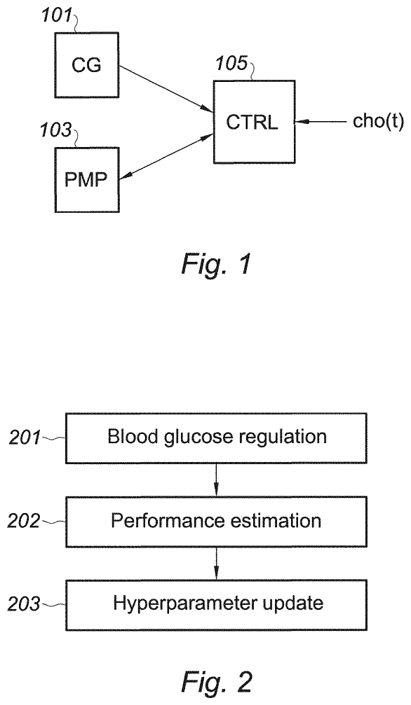

FIG. 1 schematically shows in the form of blocks an example of an embodiment of an automated system for regulating the blood glucose of a subject or of a diabetic user.

The system of FIG. 1 comprises a sensor 101 (CG) adapted to measuring the subject's blood glucose. In normal operation, sensor 101 may be permanently positioned on and inside of the subject's body, for example, at the height of their abdomen. Sensor 101 is for example a CGM-type (“Continuous Glucose Monitoring”) sensor, that is, a sensor capable of measuring, continuously or at a relatively high frequency (for example, at least once every twenty minutes and preferably at least once every five minutes) the subject's blood glucose. Sensor 101 is for example a subcutaneous blood glucose sensor.

The system of FIG. 1 further comprises an insulin injection device 103 (PMP), for example, a subcutaneous injection device. Device 103 is for example, an automatic injection device of insulin pump type, comprising an insulin reservoir connected to an injection needle implanted under the subject's skin, and the pump may be electrically controlled to automatically inject determined insulin doses at determined times. In normal operation, injection device 103 may be permanently positioned on and inside of the subject's body, for example, at the level of their abdomen.

The system of FIG. 1 further comprises a processing and control unit 105 (CTRL) connected on the one hand to blood glucose sensor 101, for example, by a wire link or by a radio (wireless) link, and on the other hand to injection device 103, for example, by wire or radio link. In operation, processing and control unit 105 is capable of receiving the data relative to the patient's blood glucose measured by sensor 101, and of electrically controlling device 103 to inject to the subject determined insulin doses at determined times. In this example, processing and control unit 105 is further capable of receiving, via a user interface, not detailed, data cho(t) representative of the time variation of the quantity of glucose ingested by the patient. Processing and control unit 105 may further be adapted to receiving possible complementary data, for example, data relative to the user's physical activity and/or state of stress, or also relative to their state of health.

Processing and control unit 105 is capable of determining the insulin doses to be injected to the patient by taking into account, in particular, the history of the blood glucose measured by sensor 101, the history of the insulin injected by device 103, and the history of glucose ingestion by the patient, as well as possible complementary data, for example, data relative to the patient's physical activity and/or state of stress. To achieve this, processing and control unit 105 comprises a digital calculation circuit (not detailed), for example comprising a microprocessor. Processing and control unit 105 is for example a mobile device carried by the patient all along the day and/or the night. As an example, processing and control unit 105 is rigidly assembled to insulin injection device 103 or to sensor 101. As a variant, processing and control unit 105 is a device independent from injection device 103 and from sensor 101, for example, a smartphone-type device.

Processing and control unit 105 is configured to implement an automated regulation method capable of comprising a plurality of distinct regulation bricks or modules respectively corresponding to distinct regulation modes.

In particular, the regulation method implemented by processing and control unit 105 may comprise a brick implementing a MPC-type (“Model-based Predictive Control”) regulation method, also called predictive control method, where the regulation of the administered insulin dose takes into account a prediction of the future trend of the patient's blood glucose over time, obtained from a mathematical model, for example, a physiological model describing the assimilation of insulin by the patient's body and its impact on the patient's blood glucose. In this operating mode, the real blood glucose data measured by sensor 101 are mainly used for purposes of calibration of the mathematical model.

The regulation method implemented by processing and control unit 105 may further comprise a brick implementing a security capping algorithm, also called hypominimizer (HM), or hypoglycemia minimizing algorithm, having the function of anticipating and of preventing imminent hypoglycemias by interrupting the insulin flow administered by device 103 and/or by recommending a glucose administration to the patient, that is, a carbohydrate ingestion. Indeed, in certain situations, the predictions made by the mathematical model of the MPC brick may occur not to be sufficiently reliable, whereby the control of insulin injection device 103 based on the predictions made by mathematical model of the MPC brick only does not enable to correctly regulate the patient's blood glucose. The hypoglycemia minimization capping algorithm enables to predict an imminent risk of hypoglycemia and, when such a risk is detected, to decrease or interrupt the flow of insulin injected to the patient, or even to recommend a glucose administration to the patient to try avoiding the hypoglycemia.

The regulation method implemented by processing and control unit 105 may further comprise a brick implementing a regulation method of decision matrix type (MD), capable of being used as a substitute to the predictive control regulation algorithm of the MPC brick, for example when it is determined that the predictions made by the mathematical model of the MPC brick are not sufficiently reliable.

The regulation method implemented by processing and control unit 105 may further comprise a post-prandial management brick (PMM) implementing a specific regulation method during phases following the meals declared by the user.

The regulation method implemented by processing and control unit 105 may further comprise a brick implementing a bolus adjustment algorithm and various sensitivity parameters, for example by using a decision tree.

The regulation method implemented by processing and control unit 105 uses a large number of parameters. Some of these parameters are fixed, that is, they cannot be modified without entirely recompiling the regulation software, which implies an interruption of the regulation to perform their update. Other parameters, called hyperparameters, may be modified on the fly, or in the moment, that is, without interrupting the regulation. Each hyperparameter has a default value, and can be adjusted by processing and control unit 105 by means of an adjustment function, between a minimum value and a maximum value.

As a non-limiting example, the regulation method may use a hyperparameter, which will be called PATIENT_HYPO_LIMIT, corresponding to a blood glucose threshold below which the user will be considered as being hypoglycemic by the hypominimizer security brick (HM). This parameter may particularly be used by the capping algorithm implemented by the HM brick to decide to interrupt the insulin flow administered by device 103 and/or to recommend a glucose administration to the user. This parameter has a default value, for example, in the order of 70 mg/dl, but may be modified without interrupting the regulation between a minimum value, for example, in the order of 60 mg/dl, and a maximum value, for example, in the order of 85 mg/dl. The setting of this parameter enables in fine to control the number of glucose administrations recommended to the patient and/or the number of interruptions of the insulin flow administered to the patient.

Another example of hyperparameter capable of being used by the regulation method is a coefficient that will be called MD_BOLUS_FACTOR hereafter, used by the decision matrix (MD) brick to determine the size of the boluses (insulin dose) to be injected to the patient at the end of a decision phase. This parameter has a default value, for example, in the order of 0.7, and may be modified without interrupting the regulation between a minimum value, for example, in the order of 0.3, and a maximum value, for example, in the order of 1.3. The setting of this parameter enables in fine to control, for a given patient, the quantity of injected insulin when the regulation is performed by the decision matrix brick.

Another example of hyperparameter capable of being used by the regulation method is an error threshold, which will be called MODEL_MISMATCH hereafter, used to estimate the reliability of the predictions made by the mathematical model of the MPC brick, and decide to switch or not from the MPC brick (predictive control regulation based on a mathematical model) to the MD brick (decision matrix regulation). More particularly, processing and control unit 105 may be configured to, during a phase of estimation of the reliability of the mathematical model of the MPC brick, calculate a digital indicator representative of the error between the blood glucose estimated from the model and the real blood glucose measured by sensor 101, and compare this indicator with threshold MODEL_MISMATCH. If the calculated error is smaller than the threshold, the regulation keeps on being implemented by the MPC predictive control regulation brick. If the calculated error is greater than the threshold, the MPC brick temporarily stops being used and the regulation is implemented by the brick of regulation by decision matrix MD. Parameter MODEL_MISMATCH has a default value, and may be modified without interrupting the regulation between a minimum value and a maximum value. The setting of this parameter eventually enables to control, for a given patient, the ratio of the time past in MPC regulation to the time past in MD regulation.

Another example of hyperparameter capable of being used by the regulation method is an inhibition period between two glucose administration recommendations, which will be called SNACK_INHIB_DURATION hereafter. It is a minimum period following a glucose administration declared by the user, during which brick HM is not authorized to recommend a new glucose administration. This parameter has a default value, and may be modified without interrupting the regulation, between a minimum value and a maximum value. The setting of this parameter eventually enables to control, for a given user, the number of glucose administrations recommended to the patient for a given time interval.

Another example of hyperparameter capable of being used by the regulation method is a value of increase of the targeted blood glucose value, applied before a physical activity to come declared by the user so as to take into account the physical activity to come in the regulation. This parameter, which will be called BEFORE_PA_TARGET_MAJORATION hereafter, has a default value, and may be modified without interrupting the regulation between a minimum value and a maximum value. The setting of this parameter eventually enables to control, for a given user, the risk of hypoglycemia linked to a physical activity.

More generally, many other hyperparameters are likely to be used in the regulation method implemented by processing and control unit 105.

According to an aspect of an embodiment, it is provided to automatically adjust the values of one or a plurality of hyperparameters, for example, one or a plurality of hyperparameters from the group comprising the above-mentioned parameters PATIENT_HYPO_LIMIT, MD_BOLUS_FACTOR, MODEL_MISMATCH, SNACK_INHIB_DURATION, and BEFORE_PA_TARGET_MAJORATION, according to one or a plurality of performance indicators of the regulation system.

For this purpose, the processing and control unit is configured to, at the end of a regulation phase implementing the hyperparameter(s) to be adjusted, calculate one or a plurality of indicators representative of the performance of the regulation system during said regulation phase, and then adjust on the fly (that is, without interrupting the regulation), the value of considered hyperparameter(s) according to the calculated performance indicators.

FIG. 2 illustrates an example of a method of automated adjustment of a blood glucose regulation method capable of being implemented by the system of FIG. 1.

The method of FIG. 2 comprises a step 201 during which the system automatically regulates the user's blood glucose. For this purpose, processing and control unit 105 implements a regulation method, for example based on one or a plurality of the above-described regulation bricks. During this regulation phase, processing and control unit 105 determines the insulin doses to be injected to the patient by taking into account, in particular, the history of the blood glucose measured by sensor 101, the history of the insulin injected by device 103, and the history of glucose ingestion by the patient, as well as possible complementary data, for example, data relative to the patient's physical activity and/or state of stress. Processing and control unit 105 further controls insulin injection unit 103 to administer the determined insulin doses to the user.

During regulation phase 201, the value of each of the hyperparameters which is desired to be adjusted is maintained constant. The duration of regulation phase 201 is selected such that each of the hyperparameters which is desired to be adjusted is used at least once, and preferably a plurality of times, during regulation phase 201. As an example, the duration of regulation phase 201 is selected to comprise, for each of the hyperparameters which is desired to be adjusted, at least 20 occurrences of an event implementing the considered hyperparameter.

The method of FIG. 2 further comprises, at the end of regulation phase 201, a step 202 of estimation of the performance of the regulation implemented at step 201. For this purpose, processing and control unit 105 calculates or determines one or a plurality of indicators representative of the performance of the regulation implemented during phase 201.

The performance indicators are for example one or a plurality of the indicators from the group comprising:
the percentage of time past in hypoglycemia or the number of passages in hypoglycemia during regulation phase 201;
the percentage of time past in hyperglycemia or the number of passages in hyperglycemia during regulation phase 201;
the percentage of time past in normoglycemia during regulation phase 201;
the variability of the blood glucose during regulation phase 201; and
the number of glucose administrations asked to the user during regulation phase 201.

It should be noted that there is meant by:
hypoglycemia, a state where the patient's blood glucose is lower than a predetermined low threshold;
hyperglycemia, a state where the patient's blood glucose is higher than a predetermined high threshold; and
normoglycemia, a state where the patient's blood glucose is between the low hypoglycemia threshold and the high hyperglycemia threshold.

The method of FIG. 2 further comprises, at the end of step 202, a step 203 of adjustment of the values of the considered hyperparameter(s), taking into account the performance indicator(s) determined at step 202. During this step, processing and control unit 105 modifies the values of the considered hyperparameter(s) according to predetermined rules, to attempt to improve the performance of the regulation system. As an example, in the case where the performance indicator comprises the percentage of time past in hypoglycemia or the percentage of time past in hyperglycemia, the adjustment of the hyperparameter(s) may attempt to decrease this percentage. In the case where the performance indicator comprises the percentage of time past in normoglycemia, the adjustment of the hyperparameters may attempt to increase this percentage. In the case where the performance indicator comprises the blood glucose variability, the adjustment of the hyperparameter(s) may aim at trying to decrease this variability. In the case where the performance indicator comprises the number of glucose administrations asked to the user for a given time period, the adjustment of the hyperparameter(s) may aim at trying to decrease this number.

As an example, in the case of the above-mentioned parameter PATIENT_HYPO_LIMIT, if it is considered at step 203 that the number of passages in hypoglycemia following recommendations or decisions based on the use of this parameter is too high, it may be provided to increase the value of threshold PATIENT_HYPO_LIMIT, for example, to increase it from 70 mg/dl to 75 mg/dl to continue the regulation.

In the case of parameter MD_BOLUS_FACTOR, if it is considered at step 203 that the number of hyperglycemias following bolus or insulin dose injections calculated based on this parameter is too high, it may be provided to increase the value of parameter MD_BOLUS_FACTOR, for example, by 10%, for the rest of the regulation.

More generally, it will be within the abilities of those skilled in the art, according to the considered hyperparameters and performance indicators, to determine the rules of automatic adjustment to be applied at step 203 to improve the performance of the regulation system.

The adjustment of the hyperparameter(s) at step 203 is performed on the fly, that is, without interrupting ht regulation, by means of an adjustment function implemented by processing and control unit 105.

Steps 201 to 203 may then be repeated, it being understood that the adjustment rules implemented at step 203 may take into account the variation of the performance indicator(s) between the successive iterations, particularly to determine whether the system performance varies in the right way.

Various embodiments and variants have been described. Those skilled in the art will understand that certain features of these embodiments can be combined and other variants will readily occur to those skilled in the art. In particular, the described embodiments are not limited to the specific examples of hyperparameters or to the specific examples of performance indicators mentioned in the present description. More generally, the provided method of automated and on-the-fly adjustment of the hyperparameters to increase the performance of the regulation system may be implemented for other hyperparameters than those mentioned hereabove, and based on other performance indicators than those indicated hereabove.

1. Blood glucose regulation system comprising a processing and control unit configured to implement an automated blood glucose regulation method,
wherein the regulation method takes into account at least one hyperparameter having a default value, the value of said at least one hyperparameter being adjustable on the fly by the processing and control unit by means of an adjustment function,
and wherein the processing and control unit is configured to, after a regulation period:
estimate the performance of the regulation method by means of at least one performance indicator; and
adjust on the fly the value of said at least one hyperparameter according to said at least one performance indicator.

2. System according to claim 1, further comprising:
a blood glucose sensor; and
an insulin injection device,
wherein the blood glucose regulation method implemented by the processing and control unit comprises the control of the insulin injection device by taking into account measurements provided by the blood glucose sensor.

3. System according to claim 1, wherein said at least one performance indicator is an indicator from the group comprising:
a percentage of time past in hypoglycemia or a number of passages in hypoglycemia during the regulation period;
a percentage of time past in hyperglycemia or a number of passages in hyperglycemia during the regulation period;
a percentage of time past in normoglycemia during the regulation period;
a quantity representative of the variability of the blood glucose during the regulation period; and
a number of glucose administrations recommended to the user during the regulation period.

4. System according to claim 1, wherein the value of said at least one hyperparameter is maintained constant by the processing and control unit during the regulation period.

5. System according to claim 1, wherein said at least one hyperparameter is used a plurality of times by the processing and control unit during the regulation period.

6. System according to claim 5, wherein said at least one hyperparameter is used at least twenty times by the processing and control unit during the regulation period.

7. System according to claim 1, wherein said at least one hyperparameter is a hyperparameter from the group comprising:
a blood glucose threshold below which the user is considered as being hypoglycemic by the processing and control unit;
a coefficient used by the processing and control unit to determine the volume of insulin doses to be injected to the user;
a tolerated threshold of error between a blood glucose prediction made by the processing and control unit by means of a mathematical model, and the real blood glucose measured by the sensor;
an inhibition period between two successive glucose administration recommendations made to the user by the processing and control unit; and
a value of increase of a blood glucose target, applied by the processing and control unit before a physical activity declared by the user.

8. System according to claim 7, wherein said at least one hyperparameter is the coefficient used by the processing and control unit to determine the volume of insulin doses to be injected to the user, and said at least one performance indicator is the percentage of time past in hyperglycemia or the number of passages in hyperglycemia during the regulation period,
and wherein, at the step of adjustment on the fly of the value of said at least one hyperparameter according to said at least one performance indicator, the processing and control unit increases the value of said coefficient if the value of said indicator is greater than a threshold.
